(19) United States
(12) Patent Application Publication
(10) Pub. No.: US 2012/0083734 A1
(43) Pub. Date: Apr. 5, 2012

(54) BALLOON CATHETER COMPRISING PRESSURE SENSITIVE MICROPARTICLES

(75) Inventors: Lee Ayres, Nijmegen (NL); Dennis Manuel Vriezema, Nijmegen (NL); Johannes Hendrikus Leonardus Hanssen, Erlecom (NL); Johannes Antonius Opsteen, Nijmegen (NL)

(73) Assignee: Encapson B.V., AJ Nijmegen (NL)

(21) Appl. No.: 13/320,734

(22) PCT Filed: May 17, 2010
(86) PCT No.: PCT/EP2010/056756

§ 371 Date: Dec. 14, 2011

(30) Foreign Application Priority Data
May 18, 2009 (EP) 09160530.3

(51) Int. Cl. A61M 29/00; A61M 31/00

(52) U.S. Cl. 604/103.02; 604/500

(57) ABSTRACT

The invention provides a solution to the above mentioned problem in that it provides a catheter balloon comprising a flexible coating on its outer surface wherein a plurality of microparticles are contained wherein said coating comprises a material selected from the group consisting of poly(N-vinyl-pirrolidone, poly(N-vinyl-pirrolidone-co-butylacrylate), poly(-vinyl pyridine), polyacrylamides, e.g. poly(N-isopropylacrylamide), poly(amido-amines), poly(ethylene imine), poly(ethylene oxide-block-propylene oxide), poly(ethylene oxide-block-propylene oxide-block-ethylene oxide), poly(styrene-block-isobutylene-block-styrene), poly(hydroxystyrene-block-isobutylene-block-hydroxystyrene), polydialkylsiloxanes, polysaccharides, polyacrylates and polyalkylmethacrylates, e.g. polymethylmethacrylate and poly(2-hydroxyethylmethacrylate) and wherein said microparticles comprise a material selected from the group consisting of polyesters, e.g. poly(lactic acid), poly(lactic-co-glycol acid), poly(glycolic acid), poly(3-hydroxybutyrate), poly(3-hydroxyvalerate), poly(3-hydroxybutyrate-co-3-hydroxyvalerate) and polycaprolactone, polyamides, polysaccharides, polyurethanes, polyalkylmethacrylates and polyacrylates, e.g. polymethylmethacrylate and poly(2-hydroxyethylmethacrylate) and wherein the microparticles comprise a pharmaceutically active compound.

### FIELD OF THE INVENTION

The present invention relates to balloon catheters, more in particular to balloon angioplasty catheters from which plaque reducing compounds can be released.

### BACKGROUND OF THE INVENTION

Balloon catheters are currently being used to open up blood vessels that are affected by plaque. It is known in the art that there are drugs that reduce or dissolve plaque. C. Herdeg et al. describe how local delivery of paclitaxel prevents restenosis (J. Am. Coll. Cardiol. 35, 1969-1976 (2000)). The delivery of drugs using a balloon catheter has previously been reported by A. Posa et al. in Coron. Artery Dis. 19, 243-247 (2008).

In U.S. Pat. No. 5,893,840 and references therein a description is given of balloon catheters capable of delivering drugs by inflating the balloon in the lumen of a blood vessel. U.S. Pat. No. 5,893,840 describes that microparticles may be coated on a balloon mounted on a catheter. When the balloon is inflated the microparticles rupture due to stretching of the coating.

This has the drawback that a part of the therapeutic compound may be flushed away by the blood stream before the balloon blocks the lumen completely. In this way, only a part of the plaque affecting compound is delivered to the desired location.

EP1981559 describes a method to coat folded balloons of balloon catheters for drug delivery.

There remains a need for a device that ensures more efficient and safe delivery of drugs to a local target in the blood vessel.

### SUMMARY OF THE INVENTION

The invention provides a solution to the above mentioned problem in that it provides a catheter balloon comprising a flexible coating on its outer surface wherein a plurality of microparticles are contained wherein said coating comprises a material selected from the group consisting of poly(N-vinyl-pirrolidone), poly(N-vinyl-pirrolidone-co-butylacrylate), poly(4-vinyl pyridine), polyacrylamides, e.g. poly(N-isopropylacrylamide), poly(amido-amines), poly(ethylene imine), poly(ethylene oxide-block-propylene oxide), poly(ethylene oxide-block-propylene oxide-block-ethylene oxide), poly(styrene-block-isobutylene-block-styrene), poly(hydroxystyrene-block-isobutylene-block-hydroxystyrene), polydialkylsiloxanes, polysaccharides, polyacrylates and polyalkylmethacrylates, e.g. polymethylmethacrylate and poly(2-hydroxyethylmethacrylate) and wherein said microparticles comprise a material selected from the group consisting of polyesters, e.g. poly(lactic acid), poly(lactic-co-glycol acid), poly(glycolic acid), poly(3-hydroxybutyrate), poly(3-hydroxyvalerate), poly(3-hydroxybutyrate-co-3-hydroxyvalerate) and polycaprolactone, polyamides, polysaccharides, polyurethanes, polyalkylmethacrylates and polyacrylates, e.g. polymethylmethacrylate and poly(2-hydroxyethylmethacrylate) and wherein the microparticles comprise a pharmaceutically active compound.

This catheter balloon may be used in a method for delivering a pharmaceutically active compound to a predestinated target in a blood vessel. For that purpose, the device is inserted into a patient in need of such a therapy, monitoring whether the device has reached its predestinated target and inflating the balloon, thereby releasing the pharmaceutically active compound.

### DETAILED DESCRIPTION OF THE INVENTION

The invention provides a catheter balloon comprising a coating that contains microparticles encapsulating a pharmaceutically active compound.

The catheter balloon may be any conventional balloon, for instance the balloons that are commercially available from for instance Abbott, Boston Scientific, Cordis and Medtronic.

The microparticle should comprise a material selected from the group consisting of polyesters, e.g. poly(lactic acid), poly(lactic-co-glycol acid), poly(glycolic acid), poly(3-hydroxybutyrate), poly(3-hydroxyvalerate), poly(3-hydroxybutyrate-co-3-hydroxyvalerate) and polycaprolactone, polyamides, polysaccharides, polyurethanes, polyalkylmethacrylates and polyacrylates, e.g. polymethylmethacrylate and poly(2-hydroxyethylmethacrylate). This provides the microparticle with the optimal brittleness so that it will rupture when the balloon is inflated and the microparticles are pressed against the blood vessel wall. Such microparticles, however, will remain intact during the inflation process and will not rupture due to shear stress as is the case with conventional microparticles contained in a conventional coating.

The shape of the microparticles may be spherical, ellipsoid, amorphous, cubical, elongated, truncated, orthorhombic or cylindrical. Their average diameter can range from 0.1 to 100 micrometer, preferably between 0.5 to 20 micrometer. Preferred are spherical hollow microparticles with a shell that is brittle.

Suitable methods for the preparation of microparticles are described in WO8808300 by E. Mathiowitz and R. S. Langer in U.S. Pat. No. 5,893,840 and in Macromol. Bioscience, 8, 991-1005 (2008) by D. Lensen et al., the disclosures of which are incorporated herein by reference.

The microparticles encapsulate a pharmaceutically active compound or a drug. This may be in the form of a solid, e.g. crystalline, semi-crystalline or amorphous material, a gel, a sol-gel, an oil, suspension, dispersion, emulsion or a solution. The type of drug may be any drug which is beneficial in treating the lumen of a blood vessel. The pharmaceutically active compound may also be a compound used in the diagnosis of a particular disease. Suitable pharmaceutically active compounds or drugs are exemplified in US2009/0054837 which is incorporated herein by reference in its entirety.

The coating of a device according to the invention consists of a material selected from the group consisting of poly(N-vinyl-pirrolidone), poly(N-vinyl-pirrolidone-co-butylacrylate), poly(4-vinyl pyridine), polyacrylamides, e.g. poly(N-isopropylacrylamide), poly(amido-amines), poly(ethylene imine), poly(ethylene oxide-block-propylene oxide), poly(ethylene oxide-block-propylene oxide-block-ethylene oxide), poly(styrene-block-isobutylene-block-styrene), poly(hydroxystyrene-block-isobutylene-block-hydroxystyrene), polydialkylsiloxanes, polysaccharides, polyacrylates and polyalkylmethacrylates, e.g. polymethylmethacrylate and poly(2-hydroxyethylmethacrylate). The combination of these coating materials with the selected materials for the microparticles provide the device with the unique property that the particles will not release their content unless they are pressed against the wall of the blood vessel.

This selection of materials provides a particularly good hydrophilic and flexible coating which lowers the friction in the lumen and can keep the microparticles adhered while the balloon is moved through the lumen and during inflation.

Suitable methods for applying a coating may be found in WO2007/089761, U.S. Pat. No. 5,893,840 and US 2009/0054837 and in WO0139811 which are incorporated by reference in its entirety herein.

The microparticles can be applied on the balloon in several ways. One approach is to apply a coating on the balloon and the microparticles are applied on the balloon while the coating is still wet. Another approach is to mix the microparticles with the coating solution before it is applied to the balloon. Yet another way is to make the microparticles adhere via electrostatic interactions. This can be achieved by coating the balloon with a charged coating and then add microparticles that are oppositely charged. The microparticles can also be covalently linked to either the surface of the device or the coating, or linked to the device or coating via physisorption or chemisorption.

The technical challenge underlying the present invention was to provide a set of materials with the right properties for coatings in combination with materials for microparticles containing a pharmaceutical compound. The microparticles at the outer surface of the balloon catheter should have sufficient resilience, flexibility and strength to keep the pharmaceutically active compound contained in the microparticles when the balloon was not inflated and also during the inflation process, whereas the microparticles should readily release their content when the balloon was pressed against the wall of a lumen such as a blood vessel.

Particularly good results in this respect were achieved when a non-rigid coating consisting of poly(N-vinylpirrolidone) was combined with microparticles consisting of poly(methyl urea). Such a combination resulted in a balloon catheter coating that releases the drug only when the balloon presses against a surface upon inflation.

Excellent results were also obtained when microparticles of polycaprolactone were used. Even better results were obtained when microparticles comprising poly(lactic acid) were used. In particular, excellent release characteristics were obtained when these microparticles additionally comprised triacetin.

When poly(lactic acid) microparticles were prepared in the presence of camphor and ammonium carbonate, such release characteristics even improved.

Particularly good results were obtained when the microparticles as described herein contained micelles comprising the pharmaceutical compound. This resulted in the best release characteristics measured. Such micelles may be introduced into the microparticles as described in example 6 or by other means known in the art.

The combination of the coating materials with the materials for the microparticles as described herein allows for the construction of balloon catheters that are coated with microparticles filled with a therapeutic substance that is released only when pressed against a surface by inflation of the balloon. The coating is flexible enough to stretch with the expanding balloon and does not rupture the microparticles.

The balloon according to the invention may be used in a method for delivering a pharmaceutically active compound to a predestinated target in a blood vessel. For that purpose, the device is inserted into a patient in need of such a therapy, monitoring whether the device has reached its predestinated target and inflating the balloon, thereby releasing the pharmaceutically active compound.

In this way stenosis and plaque formation may effectively be treated.

A catheter balloon comprising the microparticles comprising the pharmaceutically active compound according to the invention is preferably incapable of releasing the pharmaceutically active compound when the balloon is not inflated. It will also not release the pharmaceutically active compound during the inflation process. Instead, it will release its content only when pressed against the wall of the lumen wherein the balloon is inserted, such as the wall of a blood vessel.

### LEGEND TO THE FIGURES

FIG. 1a is a representation of a balloon catheter inside a lumen in the deflated state having a coating with microparticles that contain a drug.

FIG. 1b is a representation of a balloon catheter inside a lumen in the inflated state. The microparticles have released the drug when they ruptured by the pressure against the lumen wall.

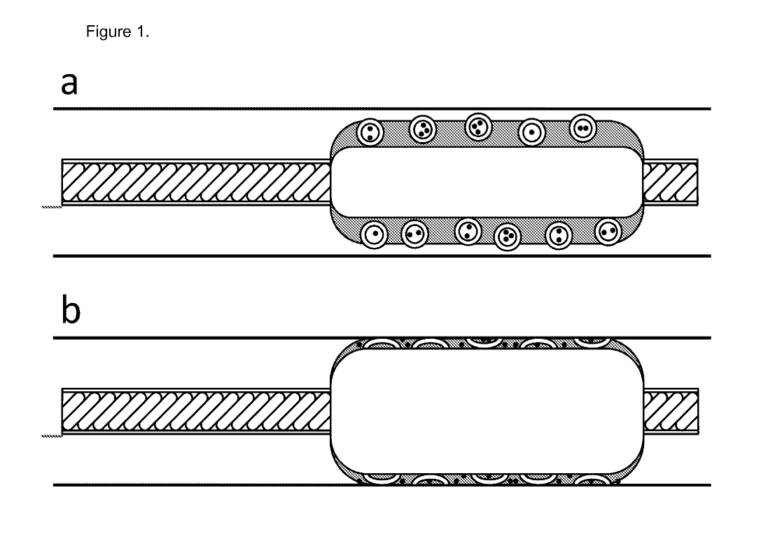

### EXAMPLE 1

Microparticles of poly(methyl urea) were prepared following the procedure described by E. N. Brown et al. in J. Microencapsulation, 20, 719-730 (2003).

In general, a suitable method for the preparation of poly(methyl urea) microparticles is to dissolve urea (5.0 g, 83 mmol), ammonium chloride (0.5 g, 9.5 mmol) and resorcinol (0.5 g, 4.5 mmol) in a 2,5% (w/w) solution of poly(ethylene-alt-maleic anhydride) in water (200 ml). The pH may be raised from 2.44 to 3.70 by dropwise addition of a 0.1 M NaOH solution and, subsequently, lowered to 3.50 using a 0.1 M HCl solution.

The aqueous solution was agitated with an Ultra-Turrax at 15,200 rpm and a few droplets of 1-octanol were added to eliminate foam formation. A slow stream of paraffin containing a minute amount of Oil-Red-O was added to form an emulsion. The high speed stirring with the Ultra-Turrax was continued for 5 minutes in order to stabilize the emulsion.

Afterwards, the emulsion was transferred to a beaker equipped with a magnetic stirring bar and formaldehyde 37% (11.6 ml, 422 mmol) was added. The reaction mixture was slowly heated (1 ° C./min) in a temperature controlled water bath to the target temperature of 55 ° C. After 4 hours the magnetic stirring and heating was ended. Once cooled to ambient temperature, the microparticles were purified by filtration and washing with distilled water.

The balloon of a Liberte™ MR catheter of Boston Scientific was coated with Labocoat® from Labo B.V. The microparticles prepared with the method described above were applied to the wet coating by spray drying. The balloon was left to dry at room temperature and atmospheric pressure.

The functioning of the coated balloon catheter was demonstrated by inserting the balloon in a clear Tygon® tube from Saint-Gobain with an inner diameter of 2.4 mm and an outer diameter of 4.0 mm. The balloon was inserted in the tube and fixed on a Zeiss Axiovert 135 TV microscope. The balloon was inflated and when the microparticles were pressed against the wall of the tube they ruptured and released their content.

### EXAMPLE 2

Poly(ε-caprolactone) microparticles containing paclitaxel and (D+)-camphor were prepared in an oil-in-water emulsion. To generate this emulsion, a solution of poly(ε-caprolatone) (400.6 mg, 0.030 mmol), D(+)-camphor (1.20 g, 7.88 mmol) and paclitaxel (80.4 mg, 0.094 mmol) in dichloromethane (8 mL) was added slowly to an aqueous solution of poly(vinyl alcohol) (4% (w/v), 80 mL) and homogenized at 6,000 rpm for 5 minutes. Subsequently, the emulsion was continually stirred for 18 hours to allow complete evaporation of dichloromethane. The microparticles were collected by centrifugation (4,000 rpm, 5 minutes) and washed with distilled water twice. The obtained capsule dispersion was flash frozen and lyophilized for 48 hours to remove all volatiles and sublime D(+)-camphor from the microparticles.

These microparticles are fully degradable and release the paclitaxel they encapsulate upon degradation. They may be incorporated in a coating on top of a catheter balloon, such as a polyurethane coating and when pressure is applied they release the encapsulated paclitaxel.

### EXAMPLE 3

A solution of poly(D,L-lactic acid) (801.5 mg, 0.067 mmol), D(+)-camphor (203.3 mg, 1.34 mmol) and paclitaxel (160.4 mg, 0.19 mmol) in dichloromethane (10 mL) was added slowly to a 5% (w/v) solution of poly(vinyl alcohol) in distilled water (100 mL) and homogenized at 4000 rpm for 5 minutes. Afterwards, the formed oil-in-water emulsion was continued stirring for 18 hours to evaporate dichloromethane and harden the microparticles. The microparticles were isolated by centrifugation (4000 rpm, 5 minutes) and washed with distilled water twice. A dispersion of the microparticles in water was flash frozen and lyophilized for 48 hours.

These microparticles are fully degradable and release the paclitaxel they encapsulate upon degradation. They may be incorporated in a coating on top of a catheter balloon such as a polyurethane coating and when pressure is applied they release the encapsulated paclitaxel. The poly(lactic acid) microparticles surprisingly released their content more exhaustively and quicker compared to polycaprolactone microparticles. Moreover, the fragmentation of the poly(lactic acid) microparticles is higher.

### EXAMPLE 4

A solution of poly(L-lactic acid) (801.1 mg, 0.080 mmol), triacetin (5 mL) and paclitaxel (162.4 mg, 0.19 mmol) in dichloromethane (20 mL) was added slowly to an aqueous poly(vinyl alcohol) solution (4% m/v) which was saturated with triacetin. Subsequently, an oil-in-water emulsion was formed by homogenization at 8,000 rpm for 5 minutes. All dichloromethane was allowed to evaporate by continuous stirring for 18 hours. The microparticles were collected by centrifugation (4,000 rpm, 5 minutes) and washed with water twice. The oil filled microparticles were air-dried for 2 days.

The presence of triacetin inside the microparticles is beneficial in the release of paclitaxel when the microparticles are disrupted upon pressing against the wall of the tube when the coated balloon is inflated. The coating is preferably a polyurethane coating The oil is pressed out of the microparticles when they are pushed against the wall of the tube which causes the paclitaxel to leave the microparticles.

### EXAMPLE 5

A 0.4 M solution of ammonium carbonate in distilled water (1 mL) was added slowly to a solution of poly(D,L-lactic acid) (801.3 mg, 0.19 mmol), D(+)-camphor (50.1 mg, 0.33 mmol) and paclitaxel (80.3 mg, 0.094 mmol) in dichloromethane (10 mL) and homogenized at 5600 rpm for 1 minute. The formed water-in-oil emulsion was poured quickly into an aqueous solution of poly(vinyl alcohol) (5% w/v, 50 mL) and homogenized at 3400 rpm for 4 minutes. The obtained double emulsion was poured into a 2% v/v solution of isopropanol in water (100 mL) and stirred for 16 hours to allow complete evaporation of dichloromethane. The microparticles were collected by centrifugation (4000 rpm, 5minutes) and washed with distilled water twice. A dispersion of microparticles in water was flash frozen and lyophilized for 48 hours. During this freeze drying process, both D(+)-camphor and ammonium carbonate sublimed and diffused out of the microparticles.

A high amount of paclitaxel is released from the microparticles when they are coated on a catheter balloon such as a balloon comprising a polyurethane coating and inflated inside a tube. The D(+)-camphor and ammonium carbonate lead to the formation of a cavity inside the microparticles which helps to break the microparticles when they are pressed against the wall of the tube.

### EXAMPLE 6

First, micelles of D-α-tocopherol poly(ethylene glycol) 1000 succinate loaded with paclitaxel were prepared by mixing a solution of paclitaxel (68.3 mg, 0.080 mmol) in ethanol (2.2 mL) with a solution of D-α-tocopherol poly(ethylene glycol) 1000 succinate (116.1 mg) in distilled water (20 mL). This mixture was subsequently sonicated for 30 minutes at room temperature and dialyzed for 20 hours against distilled water. The formed micellar solution was flash frozen and lyophilized for 18 hours.

The thus obtained solid was redispersed in distilled water (1 mL) and added slowly to a solution of poly(D,L-lactic acid) (502.8 mg, 0.042 mmol) and D(+)-camphor (50.1 mg, 0.33 mmol) in dichloromethane (10 mL). The mixture was homogenized at 6000 rpm for 1 minute after which it was poured quickly into an aqueous solution of poly(vinyl alcohol) (5% w/v, 50 mL). After 4 minutes of homogenizing at 3400 rpm, the formed emulsion was poured into a 2% v/v solution of isopropanol in water (100 mL). The emulsion was continued stirring for 18 hours to evaporate the residual dichloromethane completely. The microparticles were collected by centrifugation (4000 rpm, 5 minutes) and washed with distilled water twice. The microparticles were dispersed in water, flash frozen and lyophilized for 48 hours.

The micelles which were added to the mixture during the preparation of the microparticles is of great influence on their release profile. Release studies have shown that the microparticles prepared in this example release a high amount of paclitaxel when coated onto a balloon comprising a polyurethane coating.

1. A catheter balloon comprising a flexible coating on its outer surface wherein the flexible coating is associated with a plurality of microparticles
wherein the flexible coating comprises a material selected from the group consisting of poly(N-vinyl-pirrolidone), poly(N-vinyl-pirrolidone-co-butylacrylate), poly(4-vinyl pyridine), polyacrylamides, poly(N-isopropylacrylamide), poly(amido-amines), poly(ethylene imine), poly(ethylene oxide-block-propylene oxide), poly(ethylene oxide-block-propylene oxide-block-ethylene oxide), poly(styrene-block-isobutylene-block-styrene), poly(hydroxystyrene-block-isobutylene-block-hydroxystyrene), polydialkylsiloxanes, polysaccharides, polyacrylates, polyalkylmethacrylates, polymethylmethacrylate, and poly(2-hydroxyethylmethacrylate);
wherein the microparticles comprise a material selected from the group consisting of polyesters, poly(lactic acid), poly(lactic-co-glycol acid), poly(glycolic acid), poly(3-hydroxybutyrate), poly(3-hydroxyvalerate), poly(3-hydroxybutyrate-co-3-hydroxyvalerate), polycaprolactone, polyamides, polysaccharides, polyurethanes, polyalkylmethacrylates, polyacrylates, polymethylmethacrylate, and poly(2-hydroxyethylmethacrylate); and
wherein the microparticles further comprise a pharmaceutically active compound.

2. The catheter balloon of claim 1, wherein the microparticles do not release pharmaceutically active compound upon inflation of the catheter balloon, but release pharmaceutically active compound when the inflating catheter balloon presses against the wall of a blood vessel.

3. The catheter balloon of claim 1, wherein the microparticles comprise poly(lactic acid) or polycaprolactone.

4. The catheter balloon of claim 3, wherein the microparticles comprise poly(lactic acid) and triacetin.

5. The catheter balloon of claim 3, wherein the microparticles are prepared in the presence of camphor and ammonium carbonate.

6. The catheter balloon of claim 1, wherein the microparticles comprise micelles.

7. The catheter balloon of claim 6, wherein the micelles comprise the pharmaceutically active compound.

8. The catheter balloon of claim 7, wherein the micelles comprise D-α-tocopherol poly(ethylene glycol) 1000 succinate.

9. A method for delivering a pharmaceutically active compound to a predetermined target in a blood vessel, the method comprising:
inserting the catheter balloon of claim 1 into a subject in need of therapy with the pharmaceutically active compound,
monitoring whether the catheter balloon has reached the predetermined target, and
inflating the catheter balloon, thereby releasing the pharmaceutically active compound.
